(19) United States
(12) Patent Application Publication
(10) Pub. No.: US 2019/0059788 A1
(43) Pub. Date: Feb. 28, 2019

(54) APPARATUS AND METHOD FOR MEASURING ANATOMICAL AND PHYSIOLOGICAL PARAMETERS OF A BODY

(71) Applicant: Hayley WARREN, Little Bay (AU)

(72) Inventor: Hayley WARREN, Little Bay (AU)

(21) Appl. No.: 16/170,053

(22) Filed: Oct. 25, 2018

Related U.S. Application Data
(63) Continuation-in-part of application No. 16/036,962, filed on Jul. 17, 2018.
Application No. 16/036,962 is a continuation of application No. 14/790,015, filed on Jul. 2, 2015, now Pat. No. 10,022,069.
Application No. 14/790,015 is a continuation of application No. 13/261,037, filed on May 14, 2012, now abandoned.

(30) Foreign Application Priority Data
May 23, 2009 (AU) 2009100486
Oct. 11, 2018 (AU) 2018903846

(51) Int. Cl. A61B 5/107; G01B 9/10; A61B 5/00

(52) U.S. Cl. CPC A61B 5/1071; G01B 9/10; A61B 5/742; A61B 2560/0425

(57) ABSTRACT

An apparatus 20 for measuring an anatomical angle of a body, the apparatus 20 comprising aligning means 23 for aligning an axis 22 of the apparatus 20 with anatomical landmarks of the body, sensing means 30 for sensing an orientation of the axis 22 while the axis 22 is aligned with the landmarks, and processing means 40 for processing the sensed orientation.

Also disclosed is interactive support apparatus for measuring anatomical and physiological parameters of a body of a user and providing localised audio feedback to the user based on signals derived from measuring the anatomical and physiological parameters of the body;

said apparatus providing localised audio feedback to the user; the feedback derived from local sourced information.

### FIELD OF THE INVENTION

The present invention relates generally to an apparatus and method for measuring anatomical and physiological parameters of a body.

In one form, the anatomical parameter measured is an anatomical angle. In other forms, various other anatomical parameters and/or physiological parameters are measured and displayed. In some forms, the parameters are transmitted from the measuring apparatus.

In further particular forms, data is transmitted to the apparatus.

### BACKGROUND ART

A goniometer is an apparatus which is used by physiotherapists and the like to measure anatomical angles of a body. For example, they are often used to measure the range of angular motion of joints. It is one example of a group of devices that may take the form of a goniometer or inclinometer or petrometer.

The following patent documents disclose examples of known goniometers as well as other apparatus for measuring angles: U.S. Pat. No. 4,771,548 (Donnery), United States Patent Application Publication No. 2006/0137201 A1 (Dixon, et al.), U.S. Pat. No. 6,469,666 (Tonn), U.S. Pat. No. 7,337,751 (Lopez, et al.), U.S. Pat. No. 4,442,606 (Graham, et al.), U.S. Pat. No. 7,204,030 (Kattar), U.S. Pat. No. 7,293,363 (Emmett L. Parker), United States Patent Application Publication No. 2007/0266579 A1 (Briscoe, et al.), U.S. Pat. No. 5,163,228 (Edwards, et al.), U.S. Pat. No. 4,665,928 (Linial, et al.), U.S. Pat. No. 3,879,136 (Takeda), U.S. Pat. No. 4,665,928 (Linial, et al.), United States Patent Application Publication No. 2003/0226268 A1 (Gibson), U.S. Pat. No. 7,359,750 (Song, et al.), U.S. Pat. No. 5,253,655 (Stone, et al.), and International Patent Application No. PCT/DE1993/000891.

Many existing goniometers include a pair of aligning arms which are able to pivot relative to each other. In use, each arm is aligned with anatomical landmarks which are adjacent to the body part whose angle is being measured. For example, if a goniometer with arms is used to measure the angle between the upper part of a person's leg and the lower part of their leg, one of the arms of the goniometer will be aligned with landmarks on the upper part of the leg while the other arm is aligned with landmarks on the lower part of the leg. Once the arms have been properly aligned with the upper and lower leg parts, the angle between the arms corresponds to the angle between the leg parts and can be read off an analogue scale of the goniometer.

Goniometers of the above-described type suffer from the disadvantage that the arms are often not long enough to accurately align them with some anatomical landmarks. When this situation arises, the person using the goniometer will usually use an eye-balling approach to align the arms with the landmarks as best they can. The accuracy of the measurement which is obtained will be reduced if the arms are not properly aligned with the landmarks.

Although this problem can be overcome by extending the length of the arms (e.g. by making the arms extendable) so that they are able to reach all of the landmarks that they need to be aligned with when making a measurement, doing so would make the arms somewhat of an obstruction to a user and also make them more vulnerable to being broken or bent.

Measurement inaccuracy can also be introduced by the analogue scale of the goniometer from which the angle measurement is read. This is because the user needs to interpret the reading from the scale. Although some devices include a digital display which can address this problem, the displays tend to be small and difficult to read.

For various reasons, including those given above, many existing goniometers have poor inter therapist reliability because the variability of readings from one user to another tends to be significant. For example, there can be a 5-10 degree variance between different users. Similarly, many existing goniometers have poor intra therapist reliability because the variability of readings from the same user tends to be significant. As a consequence, measurements obtained from such goniometers are generally not taken seriously.

The inaccuracy and variance of readings obtained from existing goniometers can have significant negative consequences. For example, if a post-operative patient who has had a joint operated on must gain a certain amount of range of motion in that joint before they are able to be discharged from hospital, and if a therapist uses a goniometer to measure the range of motion of the joint to determine whether or not the patient has gained the required amount of motion, the date on which the patient is discharged will be unnecessarily delayed if the patient does have the required range of motion but, due to an inaccurate measurement obtained from the goniometer, the measured range indicates that they do not have the required range. An unnecessary delay in the patient being discharged means that the patient will needlessly be occupying a hospital bed which should be made available to someone else.

Another problem with many existing goniometers is that a user often needs to operate them with both of their hands. This means that while they are performing a measurement with such a device they do not have a hand available to assist the person on whom they are performing the measurement, or to write down the measurement which is obtained from the device.

Furthermore, many existing goniometers (particularly those which have aligning arms) are quite bulky. This bulkiness can contribute to the need for users to operate them with both hands.

Existing goniometers which have short aligning arms and/or analogue scales can also be time-consuming to use, particularly if an accurate measurement is required. This is because it can take time to properly align the arms with landmarks that the arms cannot reach, and because it can take time to properly read the scale.

In addition, at present, different sized goniometers are required to obtain measurements of different sized joints. A large goniometer is required in order to take measurements for the hip and other large joints. A small goniometer is required in order to take measurements of medium size joints and also to take measurements on the pediatric population. A finger joint goniometer is required in order to measure the angle of finger joints.

It is against this background that the present invention has been developed.

Furthermore the basic goniometer and like devices do not permit of interactive support to the user.

It is an object of the present invention to overcome, or at least ameliorate, one or more of the deficiencies of the prior art mentioned above, or to provide the consumer with a useful or commercial choice.

Other objects and advantages of the present invention will become apparent from the following description, taken in connection with the accompanying drawings, wherein, by way of illustration and example, a preferred embodiment of the present invention is disclosed.

### SUMMARY OF THE INVENTION

Accordingly in one broad form of the invention there is provided an apparatus for measuring anatomical and physiological parameters of a body; apparatus comprising a processor and a memory in communication with at least one sensor; said apparatus sensing at least one anatomical or physiological parameter of a body with which the sensor is associated thereby to output a signal comprising a measurement of the at least one anatomical or physiological parameter.

Preferably said processor is further in communication with a communications module; said communications module transmitting the signal to an external location.

Preferably the external location comprises a portable digital communications device.

Preferably the external location comprises a database.

Preferably the signal is a short range signal.

Preferably the signal is a Bluetooth signal.

Preferably the signal is a WiFi signal.

Preferably the signal is a long range signal.

Preferably the long range signal utilises TCP/IP protocol over a network.

Preferably the signal is a packet switched signal.

Preferably the database includes a AI Engine.

Preferably the portable digital communications device is in bidirectional communication with the communications module.

Preferably the database is in bidirectional communication with the communications module.

Preferably the apparatus further includes an audio output device whereby the apparatus provides interactive audio guidance to a user.

Preferably the sensor comprises one or more of accelerometer, Magnetometer, GPS Unit, EGC Sensor, Blood Pressure Sensor, Temperature Sensor, Range of Motion Sensor.

Preferably the sensor is incorporated within a cuff.

Preferably the sensor is incorporated in a cover.

Preferably the sensor is incorporated in a joint cover.

Preferably the sensor is incorporated in a knee cover.

Preferably the cover includes an inner sleeve.

Preferably the apparatus incorporates a medicine dispensing capability.

Preferably the sensor is a goniometer.

In a further broad form of the invention there is provided a method for measuring anatomical and physiological parameters of a body; said method comprising sensing at least one anatomical or physiological parameter of a body thereby to output a signal comprising a measurement of at least one anatomical or physiological parameter.

Preferably the method further includes the step of transmitting the signal to an external location.

Preferably the external location comprises a portable digital communications device.

Preferably the external location comprises a database.

Preferably the signal is a short range signal.

Preferably the signal is a Bluetooth signal.

Preferably the signal is a WiFi signal.

Preferably the signal is a long range signal.

Preferably the long range signal utilises TCP/IP protocol over a network.

Preferably the signal is a packet switched signal.

Preferably the database includes a AI Engine.

Preferably the portable digital communications device is in bidirectional communication with the communications module.

Preferably the database is in bidirectional communication with the communications module.

Preferably there is provided an audio output device whereby the method provides interactive audio guidance to a user.

Preferably the sensor comprises one or more of accelerometer, Magnetometer, GPS Unit, EGC Sensor, Blood Pressure Sensor, Temperature Sensor, Range of Motion Sensor.

Preferably the sensor is incorporated within a cuff.

Preferably the sensor is incorporated in a cover.

Preferably the sensor is incorporated in a joint cover.

Preferably the sensor is incorporated in a knee cover.

Preferably the cover includes an inner sleeve.

Preferably the apparatus incorporates a medicine dispensing capability.

Preferably the sensor is a goniometer.

Preferably the sensor is a inclinometer.

Preferably the sensor is a petrometer.

According to a further aspect of the invention there is provided an apparatus for measuring an anatomical angle of a body, the apparatus comprising aligning means for aligning an axis of the apparatus with anatomical landmarks of the body, sensing means for sensing an orientation of the axis while the axis is aligned with the landmarks, and processing means for processing the sensed orientation, wherein the processing means is able to process a sensed first orientation of the axis and a sensed second orientation of the axis to determine an angle between the axis in the sensed first orientation and the axis in the sensed second orientation.

Preferably the sensing means includes a tilt sensor for sensing the tilt of the axis, and a magnetic sensor for sensing the direction of the axis relative to a magnetic field.

Preferably the tilt sensor is able to sense the yaw, pitch and roll of the axis.

Preferably the tilt sensor is an accelerometer.

Preferably the magnetic sensor is a magnetometer.

Preferably the apparatus also comprises another aligning means for aligning another axis of the apparatus with other anatomical landmarks of the body, the aligning means are able to be pivoted relative to each other so as to vary an angle between the axes, and the processing means is able to process the sensed orientation to determine the angle between the axes.

Preferably each aligning means is a light source.

Preferably the light source is a laser.

Preferably the apparatus also comprises a display for displaying the measured angle.

Preferably the apparatus also comprises at least one switch for allowing a user to control the operation of the apparatus.

According to yet a further aspect of the invention there is provided a method for measuring an anatomical angle of a body, the method comprising the steps of:
aligning an axis with a first anatomical landmarks of the body;
sensing a first orientation of the axis while the axis is aligned with the landmarks;
aligning the axis with a second anatomical landmaks of the body;
sensing a second orientation of the axis while the axis is aligned with the landmarks; and
processing the first and second orientation to determine an angle between the axis in the sensed first orientation and the axis in the sensed second orientation.

Preferably each orientation is sensed by sensing the tilt of the axis and the direction of the axis relative to a magnetic field.

Preferably the tilt of the axis is sensed by sensing the pitch and roll of the axis.

Preferably each aligning step includes aligning a light beam with the landmarks.

Preferably the light beam is a laser beam.

Preferably the method also comprises the step of displaying the measured angle.

According to yet a further aspect of the invention there is provided an apparatus for measuring an anatomical angle of a body, the apparatus comprising aligning means for aligning an axis of the apparatus with first and second anatomical landmarks of the body, sensing means for sensing an orientation of the axis while the axis is aligned with each of the first and second landmarks, and processing means for processing the sensed orientation, wherein the apparatus is adapted to be positioned at first and second anatomical landmarks of the body for alignment of the axis of the apparatus with each of the first and second anatomical landmarks of the body for sensing the orientation of the axis at the first and second anatomical landmarks; the apparatus comprising processing means for processing the sensed orientation of the axis at the first and second anatomical landmarks to determine the angle between the axis when with the first landmark of the body and the axis when aligned to the second anatomical landmark of the body.

In yet a further broad form of the invention there is provided an interactive support apparatus for measuring anatomical and physiological parameters of a body of a user and providing localised audio feedback to the user based on signals derived from measuring the anatomical and physiological parameters of the body; said apparatus providing localised audio feedback to the user; the feedback derived from local sourced information.

Preferably the feedback is additionally derived from external sourced information.

Preferably the external sourced information is determined from the local sourced information.

Preferably the external sourced information is determined from the local sourced information and from an AI Engine; the AI engine acting on signals derived from the local sourced information and the external sourced information.

In yet a further broad form of the invention there is provided an interactive support method for measuring anatomical and physiological parameters of a body of a user and providing localised audio feedback to the user based on signals derived from measuring the anatomical and physiological parameters of the body; said method providing localised audio feedback to the user; the feedback derived from local sourced information.

Preferably the feedback is additionally derived from external sourced information.

Preferably the external sourced information is determined from the local sourced information.

Preferably the external sourced information is determined from the local sourced information and from an AI Engine; the AI engine acting on signals derived from the local sourced information and the external sourced information.

In yet a further broad form of the invention there is provided interactive support apparatus for measuring anatomical and physiological parameters of a body of a user and providing localised audio feedback to the user based on signals derived from measuring the anatomical and physiological parameters of the body; said apparatus providing localised audio feedback to the user; the feedback derived from local sourced information.

Preferably, the feedback is additionally derived from external sourced information.

Preferably, the external sourced information is determined from the local sourced information.

Preferably, the external sourced information is determined from the local sourced information and from an AI Engine; the AI engine acting on signals derived from the local sourced information and the external sourced information.

In yet a further broad form of the invention there is provided an interactive support method for measuring anatomical and physiological parameters of a body of a user and providing localised audio feedback to the user based on signals derived from measuring the anatomical and physiological parameters of the body; said method providing localised audio feedback to the user; the feedback derived from local sourced information.

Preferably, the feedback is additionally derived from external sourced information.

Preferably, the external sourced information is determined from the local sourced information.

Preferably, the external sourced information is determined from the local sourced information and from an AI Engine; the AI engine acting on signals derived from the local sourced information and the external sourced information.

### BRIEF DESCRIPTION OF THE DRAWINGS

In order that the invention may be more fully understood and put into practice, preferred embodiments thereof will now be described with reference to the accompanying drawings, in which:

FIG. 1 is a schematic block diagram of an apparatus according to a first preferred embodiment of the present invention when a battery for powering the apparatus is being charged from an external supply of electricity;

FIG. 2 is a front perspective view of the apparatus according to the first preferred embodiment of the present invention;

FIG. 3 is a rear elevation of the apparatus illustrated in FIG. 2;

FIG. 4 depicts the printed circuit board and display of the apparatus illustrated in FIG. 2 after pressing both switches of the apparatus;

FIG. 5 depicts the printed circuit board and display after both switches are pressed again;

FIG. 6 depicts the printed circuit board and display after rotating the apparatus and then pressing both switches again;

FIG. 7 depicts the printed circuit board and display after again pressing both switches;

FIG. 8 is a flowchart of a method of operating the apparatus illustrated in FIGS. 1 to 7;

FIG. 9 depicts an apparatus according to a second preferred embodiment of the present invention being used to measure an anatomical angle at a person's knee joint;

FIG. 10 depicts an apparatus according to a third preferred embodiment of the present invention when the lasers of the apparatus are activated;

FIG. 11 depicts the three main steps in operating the apparatus illustrated in FIG. 10 to measure an anatomical angle at a person's knee joint;

FIG. 12 is a schematic block diagram of an apparatus according to a fourth preferred embodiment of the present invention;

FIG. 13 is a front elevation of the apparatus according to the fourth preferred embodiment of the present invention;

FIG. 14 is a flowchart of a method of operating the apparatus illustrated in FIGS. 12 and 13.

FIG. 15 is a block schematic diagram of a further embodiment.

FIG. 16 is a block schematic diagram of yet a further embodiment.

### BEST MODE(S) FOR CARRYING OUT THE INVENTION

In the figures, like features of the described preferred embodiments of the present invention have been referenced with like reference numerals.

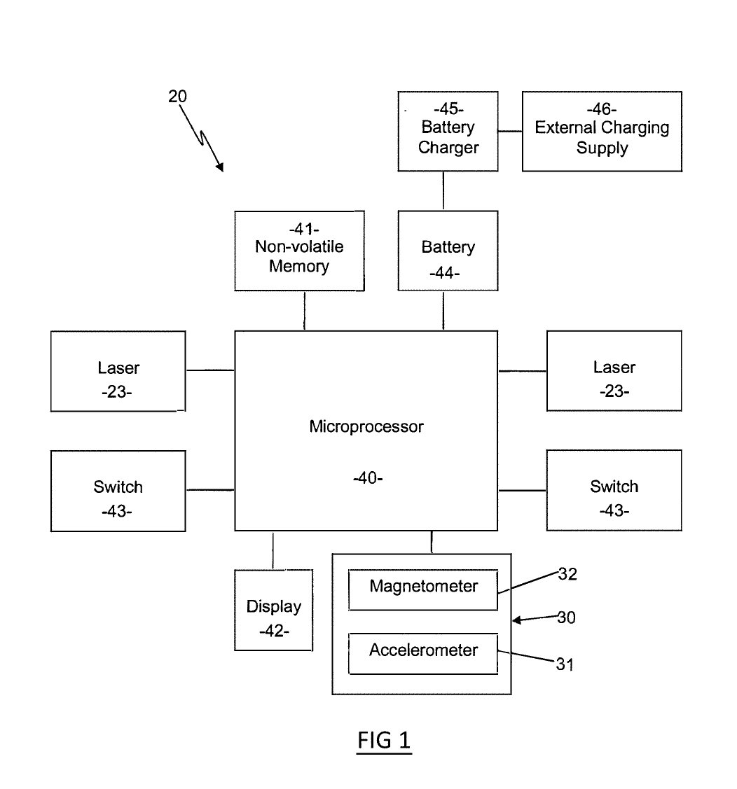

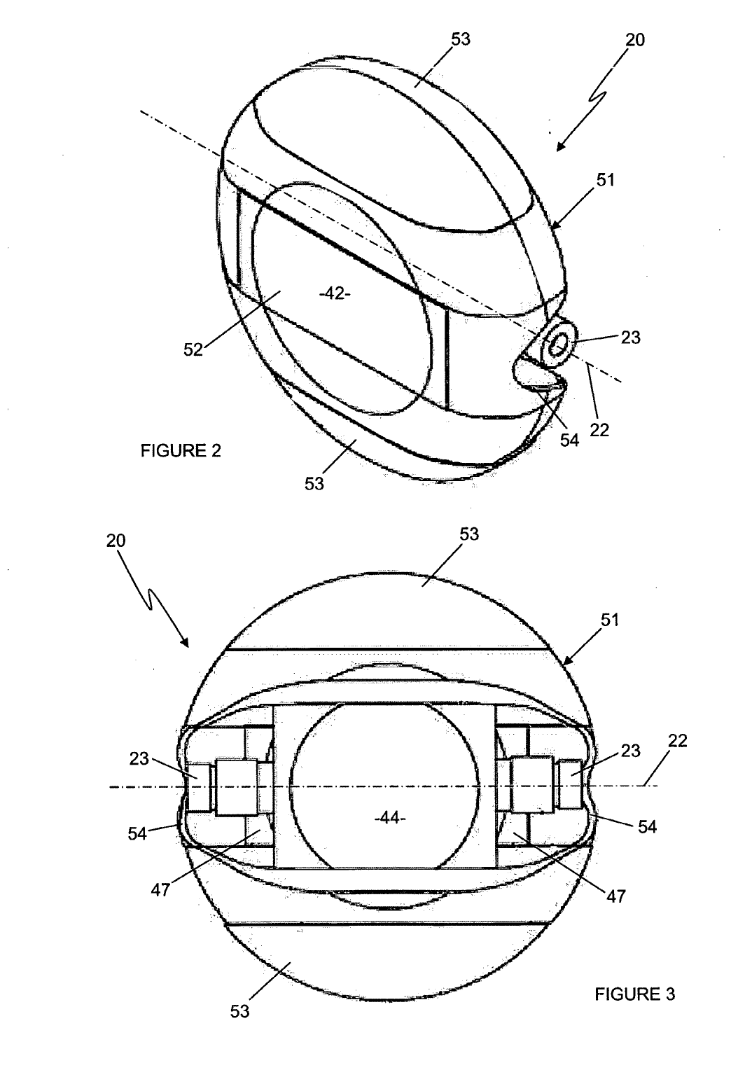

Referring to FIGS. 1 to 4, there is depicted an apparatus 20 according to a first preferred embodiment of the present invention. Apparatus 20 is for measuring an anatomical angle of a body such as a human body. Apparatus which are used for this purpose are usually referred to as goniometers by physiotherapists and the like.

Apparatus 20 includes a pair of aligning or alignment means that are each in the form of a solid-state laser 23. Lasers 23, which are diametrically opposite each other, are for aligning an axis 22 of the apparatus 20 with anatomical landmarks of a body. Each Laser 23 is able to emit a respective focused or collimated light beam in the form of a laser beam. The lasers 23 emit the laser beams outwardly from the apparatus 20 such that the laser beams are co-linear with each other and the axis 22 of the apparatus 20. Lasers 23 are preferably low-power lasers so that they do not pose a health risk.

A sensing means 30 of the apparatus 20 is a three-dimensional orientation sensor which is able to sense a first orientation of the apparatus 20 and, hence, the axis 22 in three dimensions, while the axis 22 is aligned with first anatomical landmarks of the body, and is also able to sense a second orientation of the apparatus 20 and, hence, the axis 22 in three dimensions, while the axis 22 is aligned with second anatomical landmarks of the body. Sensing means 30 includes a tilt sensor which is in the form of an accelerometer 31, and a magnetic sensor which is in the form of a magnetometer 32. Sensing means 30 is able to provide stable measurement/sensing of the three-dimensional orientation of an object in space, regardless of the object's orientation.

Accelerometer 31 is able to sense the tilt of the apparatus 20 and, hence, the axis 22. In particular, accelerometer 31 is able to sense the pitch (i.e. up/down position) and roll (i.e. side to side rocking position) of the apparatus 20 and, hence, the axis 22.

Magnetometer 32 is able to sense the yaw (rotational position about a vertical axis) of the apparatus 20 and, hence, the axis 22 relative to a magnetic field such as the earth's magnetic field.

The three-dimensional orientation of the apparatus 20 and axis 22, including the pitch, roll and yaw of the apparatus 20 and axis 22, which is sensed by the sensing means 30 is able to be output by the sensing means 30 to a processing means which is in the form of a microprocessor 40. The sensed three-dimensional orientation is output by the sensing means 30 in the form of three-dimensional coordinates. In addition to the accelerometer 31 and magnetometer 32, sensing means 30 includes interfacing circuitry (not depicted) for interfacing the accelerometer 31 and the magnetometer 32 with the microprocessor. Microprocessor 40 is able to determine an angle between the axis 22 in the sensed first orientation and the axis 22 in the sensed second orientation by processing the output of the sensing means 30 in accordance with computer-readable instructions which are able to be read by the microprocessor 40 and which are stored on storage means which is in the form of a non-volatile memory 41. Microprocessor 40 is able to output the determined angle to a liquid-crystal display (LCD) 42 which is then able to display the angle numerically in units of degrees. LCD 42 has a height of 32.41 mm, and a width of 61.82 mm.

Suitably pressing a pair of pushbutton switches 43 enables a user to control the operation of the microprocessor 40 and, hence, the apparatus 20.

Apparatus 20 is powered by a battery 44. The battery 44 may for example be a long-life lithium battery. The battery 44 can be charged by a battery charger such as a Universal Serial Bus (USB) charger 45 which is connected to an external charging supply of electricity 46.

Lasers 23, sensing means 30 including the accelerometer 31 and magnetometer 32, microprocessor 40, non-volatile memory 41, LCD 42, and switches 43 are all mounted on a printed circuit board (PCB) 47. PCB 47 is generally circular and has a radius of curvature of 30 mm. PCB 47 includes a pair of diametrically opposite recesses 48 for receiving the lasers 23. A respective pair of contact prongs 49 is secured to the PCB 47 and extends into each recess 48 from the PCB 47. Each pair of contact prongs 49 plugs into a respective slot (not depicted) in a socket (not depicted) of each laser 23.

PCB 47 is housed within a hollow disc-shaped housing 51. Housing 51 includes an opening or window 52 through which the measured angle which is displayed by LCD 42 can be viewed. It also includes a pair of buttons 53 which are located on diametrically opposite sides of the housing 51. Each button 53 operatively engages with a respective switch 43 such that the switch 43 is able to be operated by pressing the button 53. Each laser 23 is located in the housing 51 such that the lasers 23 do not protrude from the housing 51. Also, each laser 23 is located adjacent to a respective recess 54 in the housing 51 such that the housing 51 does not obstruct the laser beams which are emitted by the lasers 23.

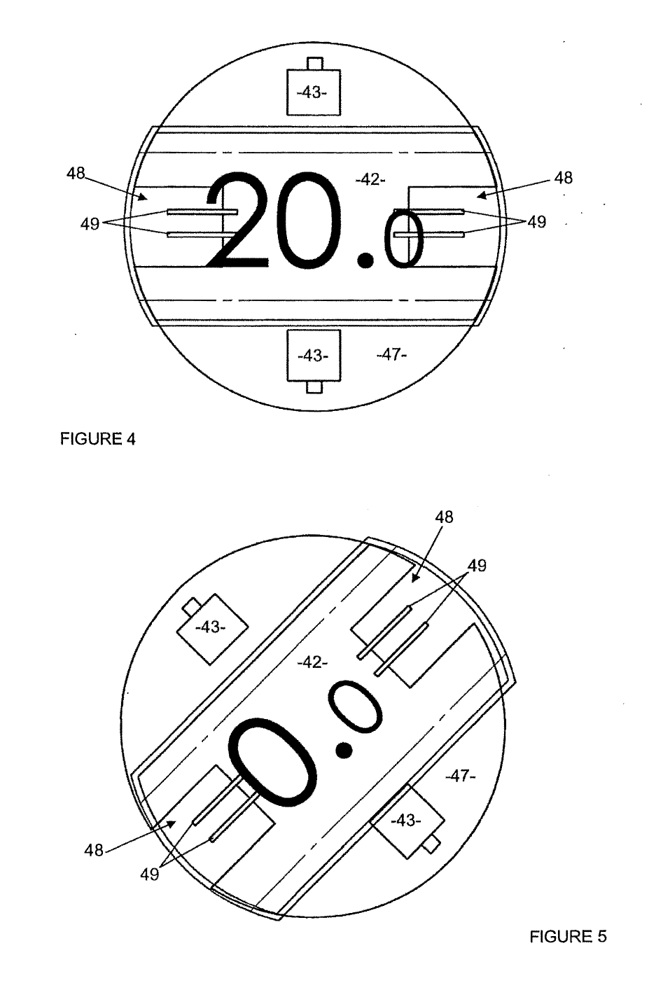

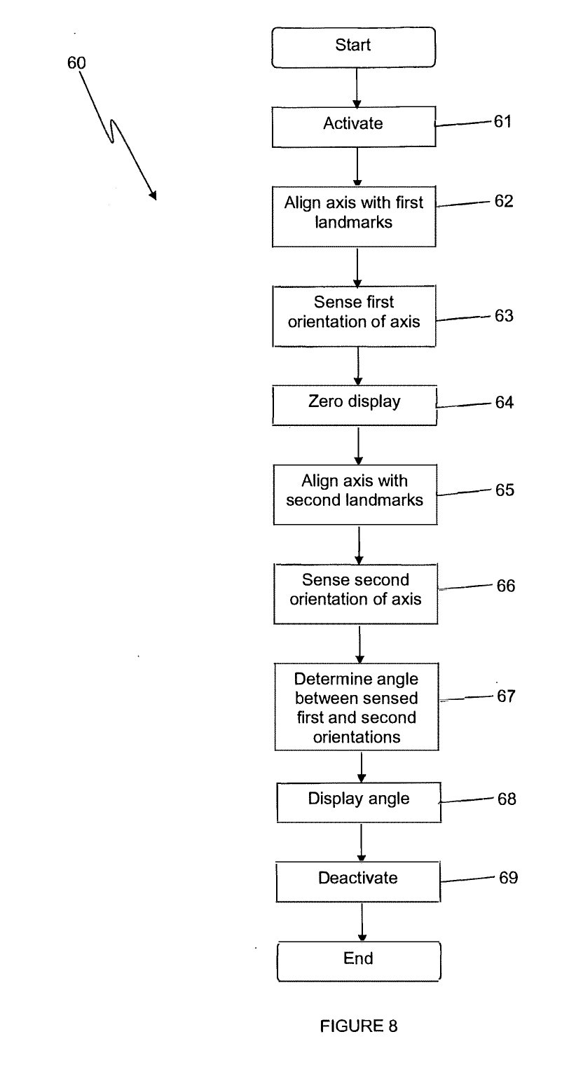

With reference to FIGS. 4 to 7, and FIG. 8 which depicts a flowchart of a method 60 of operating the apparatus 20, the apparatus 20 may be used to measure an anatomical angle of a body joint by firstly performing an activation step 61. The activation step 61 involves a user pressing both buttons 53 simultaneously with to activate (i.e. turn on) the apparatus 20. Once the buttons 53 have been pressed, both of the lasers 23 emit a laser beam, and the LCD 42 displays a numeric value in the range of 0-180 degrees. The value which is displayed by the LCD 42 depends on the orientation of the apparatus 20 and axis 22 at the time, and will change dynamically in response to a change in the orientation of the apparatus 20 and axis 22. FIG. 4 depicts the LCD 42 displaying a value of 20.0 degrees shortly after activation of the apparatus 20.

The next step of the method 60 is step 62 which is to align the laser beams which are emitted by the lasers 23 with first anatomical landmarks of the body which the joint forms part of. The laser beams are aligned with the first anatomical landmarks such that each laser beam intersects a respective one of the first anatomical landmarks. If the apparatus 20 is being used to measure a range of angular motion of a joint of a limb, the laser beams are initially aligned with landmarks at a distal end or part of the limb i.e. below the joint. For example, when measuring shoulder flexion when the patient is in supine, the apparatus 20 is positioned so that the laser beams align with the landmarks of the greater trochanter of the hip, along the line of the trunk to the mid axilla.

The landmarks which the laser beams are aligned with may be non-joint landmarks and/or joint landmarks (e.g. landmarks indicating the axis of a joint). The particular landmarks which are used by physiotherapists and the like to measure anatomical angles are well-known in the art and will not be described at length here.

Step 63, which follows step 62, involves sensing a first orientation of the apparatus 20 and axis 22 with the sensing means 30, and then capturing that information with the microprocessor 40. The first orientation of the apparatus 20 and axis 22 is the orientation that the apparatus 20 and axis 22 have when the laser beams emitted by the lasers 23 are aligned with the first anatomical landmarks of the body. Pressing both buttons 53 simultaneously while the laser beams emitted by the lasers 23 are aligned with the first anatomical landmarks causes the first orientation which is sensed by the sensing means 30 to be captured by the microprocessor 40.

Following step 63, the apparatus 20 automatically performs step 64 which is to zero the numeric value which is displayed by the LCD 42 so that the LCD will display a value of 0.0 degrees while the apparatus 20 and axis 22 are in the first orientation. FIG. 5 depicts the LCD 42 displaying a value of 0.0 degrees after the first orientation of the apparatus 20 and axis 22 have been detected, and after the numeric value displayed by the LCD 42 has been zeroed.

In other embodiments, the numeric value which is displayed by the LCD 42 may be set to zero at the time that the apparatus 20 is activated.

The next step of the method 60 is step 65 which is to align the laser beams which are emitted by the lasers 23 with second anatomical landmarks of the body which the joint forms part of. The laser beams are aligned with the second anatomical landmarks such that each laser beam intersects a respective one of the second anatomical landmarks. If the apparatus 20 is being used to measure a range of angular motion of a joint of a limb, the laser beams are aligned with landmarks at a proximal end or part of the limb (i.e. above the joint) after movement of the joint. For example, when measuring shoulder flexion, the apparatus 20 is positioned so that the laser beams align with the landmarks of the mid axilla to the medial epicondyle on the humerus.

Step 66, which follows step 63 involves sensing a second orientation of the apparatus 20 and axis 22, and capturing that information. The second orientation of the apparatus 20 and axis 22 is the orientation that the apparatus 20 and axis 22 have when the laser beams emitted by the lasers 23 are aligned with the second anatomical landmarks of the body. Pressing both buttons simultaneously while the laser beams emitted by the lasers 23 are aligned with the second anatomical landmarks causes the second orientation to be captured from the sensing means 30 by the microprocessor 40.

Step 67 of the method 60 occurs automatically after step 66. In step 67, the microprocessor 40 processes the sensed first orientation and the sensed second orientation to determine what the measured anatomical angle of the joint is.

The sensed first orientation includes a vector A (i.e. Axyz) which is an initial reference position. Vector A is sensed by the magnetometer 32, and includes pitch and roll angles which are sensed by the accelerometer 31. The sensed second orientation includes a vector B (i.e. Bxyz) which is a final reference position. Vector B is sensed by the magnetometer 32, and includes pitch and roll angles for vector B which are sensed by the accelerometer 31. Microprocessor 40 processes the sensed first orientation and the sensed second orientation by transposing vector A and Vector B to the horizontal plane so that vector A (i.e. Axyz) becomes vector A′xy, and so that vector B (i.e. Bxyz) becomes vector B′xy. The microprocessor 40 then determines the angle between transposed vector A′xy and transposed vector B′xy, and this is the angle which is displayed by the LCD 42 at step 68. A similar method is used by a compass system disclosed in a published article which is entitled “Applications of Magnetic Sensors for Low Cost Compass Systems”, by Michal J. Caruso of Honeywell, SSEC, and which is incorporated herein it its entirety by reference.

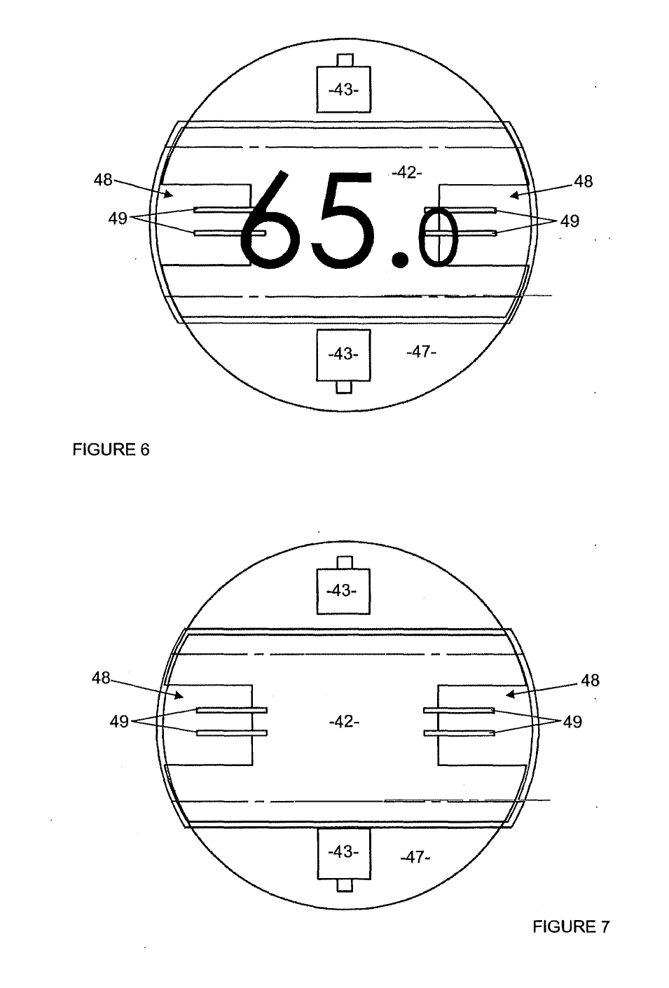

After determining the anatomical angle of the joint, the next step which is performed is step 68 which involves the microprocessor 40 controlling the LCD 42 to display this information numerically and in units of degrees. The measured anatomical angle is displayed to an accuracy of a single decimal place. This information remains display by the LCD 42 irrespective of whether or not the apparatus 20 is subsequently moved. In addition, the lasers 23 are turned off so that they no longer emit laser beams. FIG. 6 depicts the LCD 42 displaying a measured angle of 65.0 degrees.

The next step in the method is step 69 which involves the user pressing both buttons 53 so that the apparatus 20 is deactivated (i.e. turned off). FIG. 7 depicts the blank display 42 of the apparatus 20 after it has been turned off.

It will be appreciated that the sensing means 30 is not limited to including the accelerometer 31 and magnetometer 32 to sense the orientation of the axis 22 in three-dimensions, and that the sensing means 30 could use other devices/instruments to sense the three-dimensional orientation of the axis 22. For example, the sensing means 30 could use electrolytic (fluid) based tilt sensors, gimbaled mechanical structures, gyroscopes, magnetic compasses to sense the orientation of the axis 22.

The apparatus 20 may be used to measure the anatomical angle of small joints (e.g. finger joints) or the small joints of infants and the like in pediatric settings by selecting a small joint mode of operation of the apparatus by suitably pressing the buttons 53. In the small joint mode, only one of the lasers 23 is activated.

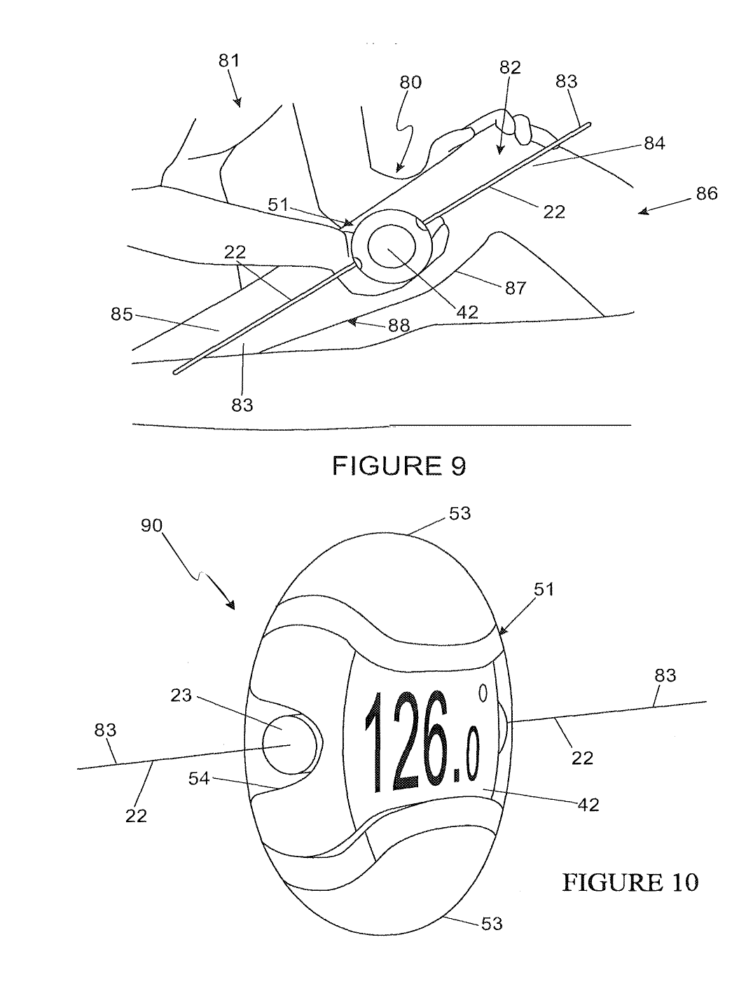

Referring to FIG. 9, an apparatus 80 according to a second preferred embodiment of the present invention is being used by a user 81 to measure an anatomical angle of a patient's right knee joint 82. Apart from some minor cosmetic differences, apparatus 80 is identical to the apparatus 20. User 81 is shown aligning laser beams 83 which are emitted by the lasers (not depicted) of the apparatus 80 with anatomical landmarks 84, 85 of the patient's right leg 86 while holding the apparatus 80 in their right hand 87 adjacent to the patient's lower right leg 88. The apparatus 80 is initially placed on the anatomical bone of the lower right leg 88 to allow intersection of the laser beams 83 with the knee joint 82 and the proximal landmark 84 of the knee 82. Laser beams 83 are co-linear with an axis 22 of the apparatus 80.

FIG. 10 depicts an apparatus 90 according to a third preferred embodiment of the present invention which is also identical to the apparatus 20 apart from some minor cosmetic differences. The lasers 23 of the apparatus 90 are shown emitting laser beams 83, and the LCD 42 of the apparatus 90 is shown displaying an angular reading of 126.0 degrees. A degrees symbol ‘°’ is displayed on the LCD 42 at the right-side of the angular reading to make it clear to a person reading the LCD 42 that the angular reading displayed thereon is in units of degrees. Laser beams 83 are co-linear with an axis 22 of the apparatus 90.

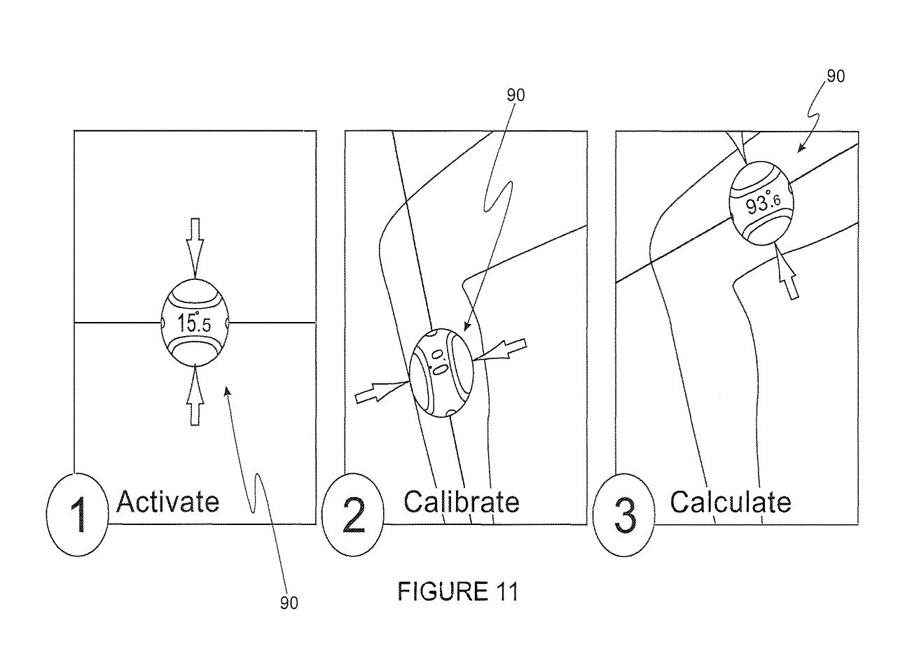

FIG. 11 depicts the three main steps in operating the apparatus 90 to measure an anatomical angle at a person's knee joint. Those steps are:

Activate;

Calibrate; and

Calculate.

In the Activate step, the apparatus 90 is activated (i.e. turned on) by pressing the buttons 53. When the apparatus 90 is activated, the LCD 42 and the lasers 23 are activated.

In the Calibrate step, the laser beams 83 are aligned with landmarks of the lower part of the leg, and then buttons 53 are pressed again to zero the reading which is displayed by the LCD 42.

In the Calculate step, the laser beams 83 are aligned with landmarks of the upper part of the leg, and then buttons 53 are pressed again so that the apparatus 90 calculates the angle between the laser beams 83 when they are aligned with the landmarks of the lower part of the leg and the laser beams 83 when they are aligned with the landmarks of the upper part of the leg.

If a user holds down the two buttons 53 for at least 5 seconds, a menu (not depicted) is displayed by the LCD 42. The menu includes a heading “Instructions for application 1, 2, 3 use”, and includes the following items:

Landmark review

Small joint mode

The menu items can be scrolled through by pressing the buttons 53. To scroll up through the menu items, the topmost button 53 is pressed. To scroll down through the menu items, the bottommost button 53 is pressed. To select a menu item, the both buttons 53 are held down for at least 5 seconds.

Selecting menu item 1—Landmark review causes a submenu to be displayed by the LCD 42. The submenu includes a list of joints (e.g. shoulder, elbow, wrist, etc.) which can be measured and whose angular measurements can be reviewed.

Selecting menu item 2—Small joint mode results in only one of the lasers 23 being activated. This means that the lasers 23 can work independently of each other. Small joint mode is used when measuring an anatomical angle of a small joint. When measuring an anatomical angle of a small joint (e.g. a finger joint), both lasers 23 are unable to be used in the manner described previously because the apparatus 90 is too large and would cover the landmarks which the laser beams 83 need to be aligned with. In the small joint mode, only one of the lasers 23 is activated, and the laser beam 83 which it emits is aligned with the landmarks.

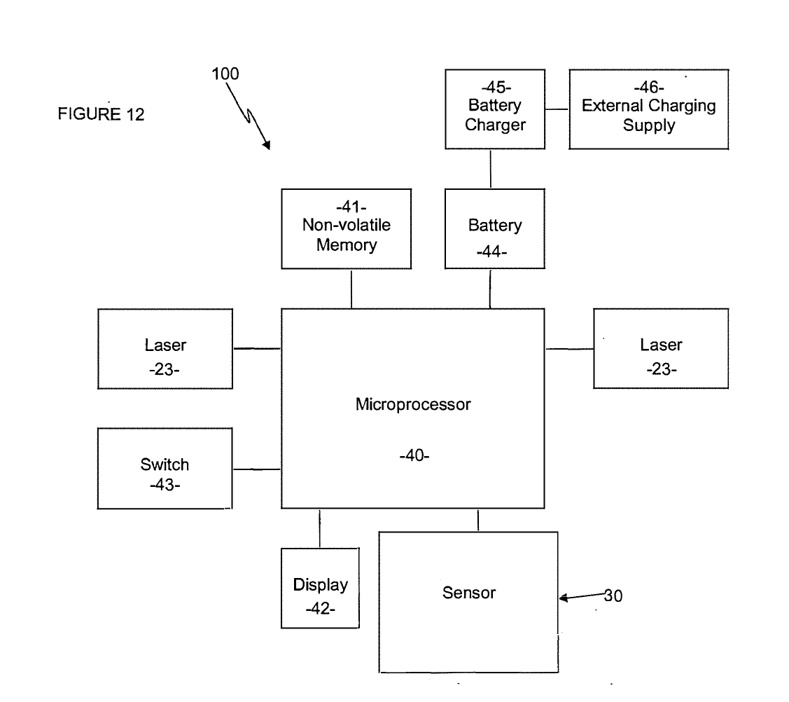

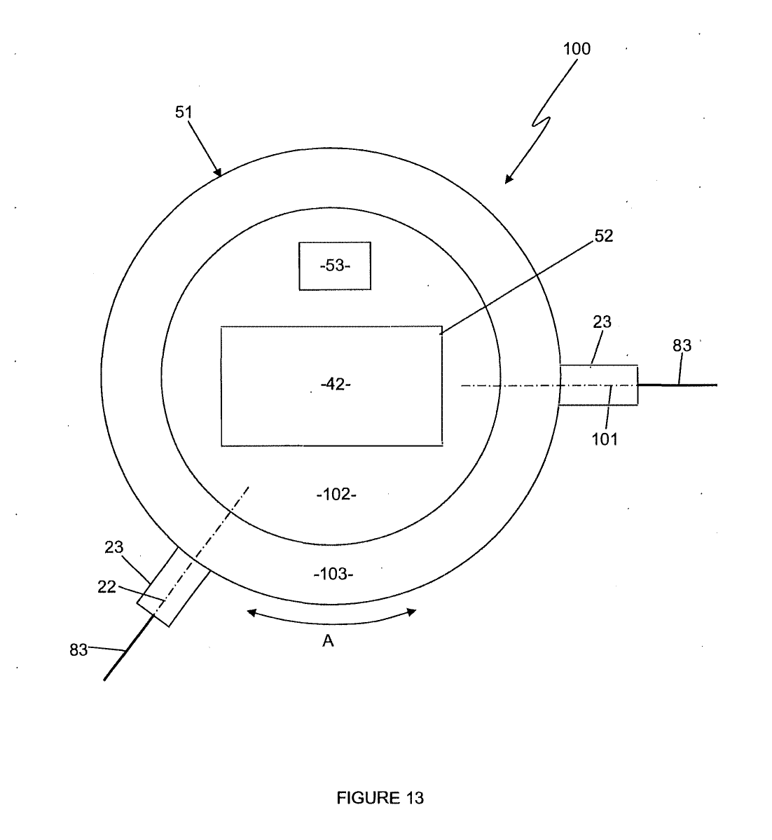

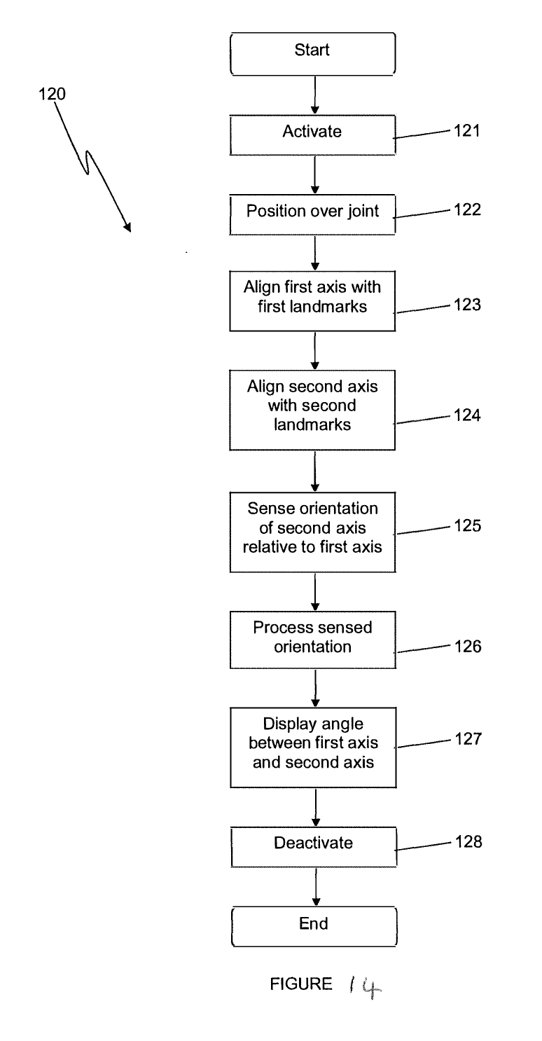

Referring to FIGS. 12 and 13, there is depicted an apparatus 100 according to a fourth preferred embodiment of the present invention. Apparatus 100 is for measuring an anatomical angle of a body such as a human body. Apparatus which are used for this purpose are usually referred to as goniometers by physiotherapists and the like.

Apparatus 100 includes an aligning or alignment means that are each in the form of a solid-state laser 23. Lasers 23 are for aligning a first axis 101 of the apparatus 100 with first anatomical landmarks of a human body, and for aligning a second axis 22 of the apparatus 100 with second anatomical landmarks of the body. Lasers 23 are each able to emit a respective focused or collimated light beam in the form of a laser beam 83. The lasers 23 emit the laser beams 83 outwardly from the apparatus 100 such that one of the laser beams 83 is co-linear with the first axis 101, and such that the other laser beam 83 is co-linear with the second axis 22. Lasers 23 are preferably low-power lasers so that they do not pose a health risk.

Apparatus 100 also includes a hollow disc-shaped housing 51 which is approximately 50 mm in diameter and which includes an inner circular part 102 which one of the lasers 23 is fixed relative to, and an outer circular part 103 which the other laser 23 is fixed relative to. The outer part 103 is able to rotate relative to the inner part 102 in either a clockwise or an anticlockwise direction as indicated by the arrow ‘A’ in FIG. 13. The laser 23 which is fixed relative to the inner part 102 is the laser 23 which is for aligning the first axis 101 with first anatomical landmarks of the body, and the laser 23 which is fixed relative to the outer part 103 is the laser 23 which is for aligning the second axis 22 with second anatomical landmarks of the body. The angle between the lasers 23 and, hence, the axes 22 and 101 can be varied by rotating the outer part 103 relative to the inner part 102. The outer part 103 can be rotated relative to the inner part through 360 degrees.

A sensing means 30 of the apparatus 100 is able to sense an orientation of the second axis 22 relative to the first axis 101. In particular, sensing means 30 is able to determine the angular orientation of the second axis 22 relative to the first axis 101.

The sensed orientation is able to be output by the sensing means 30 to a processing means 40 which is in the form of a microprocessor 40. Microprocessor 40 is able to determine the angle between the first axis 101 and the second axis 22 by processing the sensed orientation which is output by the sensing means 30 in accordance with computer-readable instructions which are able to be read by the microprocessor 40 and which are stored on storage means which is in the form of a non-volatile memory 41. Microprocessor 40 is able to output the determined angle to a liquid-crystal display (LCD) 42 which is then able to display the angle numerically in units of degrees. The angle is displayed to the nearest degree. LCD 42 can display a maximum angle of 360 degrees and a minimum angle of 0 degrees.

A pushbutton switch 43 enables a user to control the operation of the microprocessor 40 and, hence, the apparatus 100.

Apparatus 100 is powered by a battery 44. The battery 44 may for example be a long-life lithium battery. The battery 44 can be charged by a battery charger such as a Universal Serial Bus (USB) charger 45 which is connected to an external charging supply of electricity 46.

Lasers 23, sensing means 30, microprocessor 40, non-volatile memory 41, LCD 42, and switch 43 are all housed within the housing 51. Housing 51 includes an opening or window 52 through which the measured angle which is displayed by LCD 42 can be viewed. It also includes a button 53 which is located on the front of the housing 51 adjacent to the LCD 42. Button 53 operatively engages with the switch 43 of the apparatus 100 such that the switch 43 is able to be operated by pressing the button 53. Although, for clarity, the lasers 23 are shown protruding from the housing 51, they may alternatively be located in the housing 51 such that they do not protrude from the housing 51 and such that the housing 51 does not obstruct the laser beams 83 which are emitted by the lasers 23.

With reference to FIGS. 12 and 13, and also FIG. 14 which depicts a flowchart of a method 120 of operating the apparatus 100, the apparatus 100 may be used to measure an anatomical angle of a body joint by firstly performing an activation step 121. The activation step 121 involves a user pressing the button 53 to activate (i.e. turn on) the apparatus 100. Once the button 53 has been pressed, the lasers 23 emit laser beams 83, and the LCD 42 displays a numeric value in the range of 0-360 degrees. The value which is displayed by the LCD 42 depends on the orientation of the axis 22 relative to the axis 101 at the time, and will change dynamically if the orientation of the axis 22 relative to the axis 101 is altered by rotating the outer part 103 of the housing 51 relative to the inner part 102 of the housing. Rotating the outer part 103 relative to the inner part 102 changes the orientation of the axis 22 relative to the axis 101 by changing the angle between the axes 22, 101.

The next step of the method 120 is step 122 which is to position the housing 51 over the joint so that the laser beams 83 can be aligned with predetermined anatomical landmarks which are located above and below the joint.

At step 123 the laser beam 83 which is co-linear with the first axis 101 is aligned with first anatomical landmarks. The laser beam 83 is aligned with the first anatomical landmarks such that the laser beam 83 intersects each one of the first anatomical landmarks.

At step 124, the laser beam 83 which is co-linear with the second axis 22 is aligned with second anatomical landmarks. The laser beam 83 is aligned with the second anatomical landmarks such that the laser beam 83 intersects each one of the second anatomical landmarks.

Aligning the laser beams 83 with the first and second anatomical landmarks may require adjustment of the angle between the axes 22, 101 by suitably rotating the outer part 103 of the housing 51 relative to the inner part 102 of the housing 51.

At step 125, the sensing means 30 senses the orientation of the second axis 22 relative to the first axis 101 while those axes 22, 101 are aligned with the second and first anatomical landmarks, respectively. At step 126, the microprocessor 40 processes the sensed orientation to determine the angle between the first axis 101 and the second axis 22 and controls the LCD 42 to display the angle between the first axis 101 and the second axis 22.

At step 128, the apparatus 100 is deactivated (i.e. turned off) by pressing the button 53.

By employing lasers 23 instead of arms or the like as aligning means assists to make the apparatus described above more compact compared to existing goniometers which have long arms for alignment purposes.

The various apparatus described above may be used to measure the range of motion of an anatomical joint.

A user may operate the various apparatus described above using only one of their hands. This enables the user to use their free hand to assist a patient if need be, or to record the angular measurement which is obtained using the apparatus.

The lasers 23 allow the various axes of the apparatus described above to be accurately aligned with anatomical landmarks. This together with the automatic and accurate sensing, processing and display of the orientation of the various axes enables the apparatus to provide an accurate angular measurement which is more likely to be replicated between multiple measurements by the same user or measurements by different users. Consequently, the apparatus has an improved inter rater/therapist reliability and improved intra rater/therapist reliability compared to prior art devices.

In addition to being able to take accurate measurements which can be repeated by the same or different users, the apparatus are fast and easy to use.

The apparatus are able to measure anatomical angles irrespective of the orientation of the angle which is to be measured. Thus, it does not matter whether the angle to be measured lies in a horizontal plane, a vertical plane, or something in between. Consequently, the apparatus are able to be used to measure an anatomical angle of a person irrespective of the position of the person or the part of their body being measured.

The LCDs of the apparatus are sufficiently large to clearly display the angular measurement which is obtained using the apparatus.

The buttons 53 may be used to scroll through and select items from a menu which may be displayed by the LCD 42. The menu items may relate to configuring or selecting functions that the apparatus is able to perform. For example, the buttons 53 may be used to put the apparatus in to a normal measuring mode where laser beams 83 are emitted by both lasers 23, the small joint mode discussed earlier, or to select a landmark revision function. The LCD 42 may display instructions for to assist in operating the apparatus.

The housing 51 of the apparatus is preferably made from a material such as polycarbonate and aluminium for comfort and migration to the user, as well as for aesthetics and durability of the apparatus.

The battery charger 45 is not limited to being a USB battery charger and may be any sort of battery charger.

The display 42 may be a touch screen which can be used by a user to operate the apparatus. For example, the touch screen may display various options related to the operation of the apparatus, and the user may select any of those options by appropriately touching the touch screen.

### Further Embodiments

### Remote Communication Capability (H2)

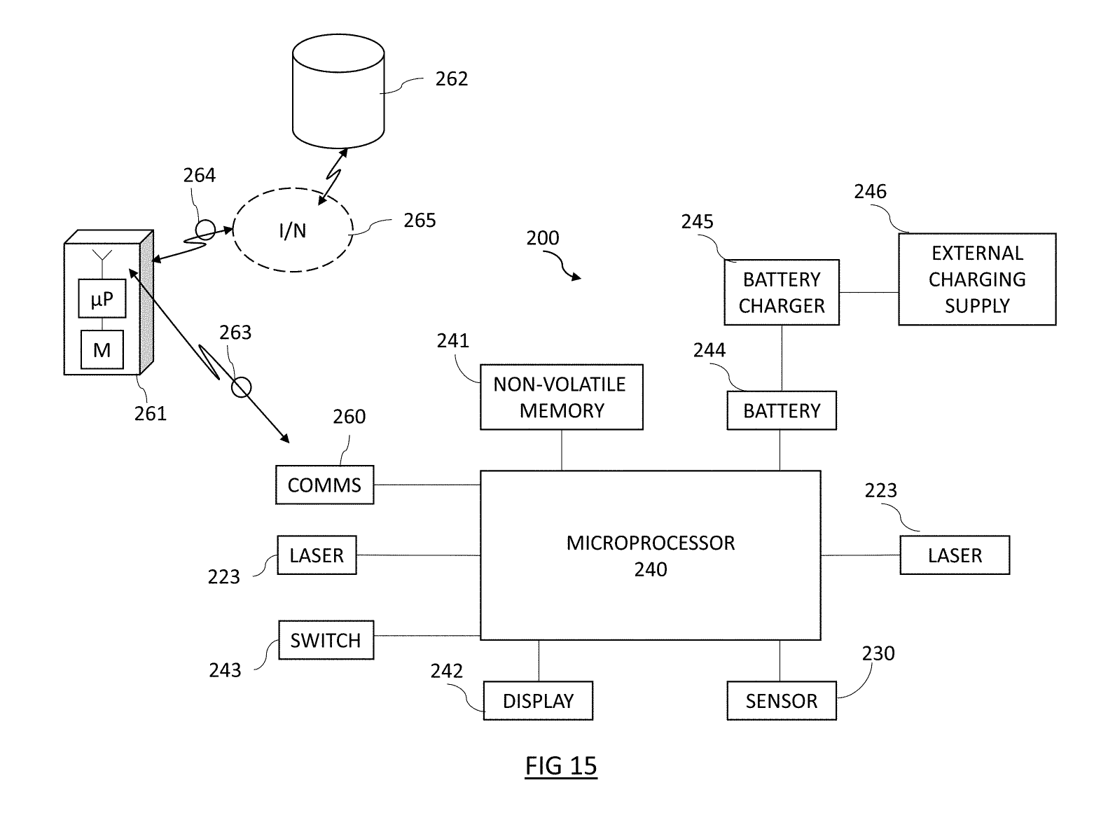

With reference to FIG. 15, there is illustrated apparatus 200 in accordance with a further embodiment of the present invention wherein like components are numbered as for earlier embodiments except in the 200 series.

In one form the apparatus 200 may perform the function of one or more of a goniometer, petrometer and inclinometer.

A petrometer is utilised to measure vertical and horizontal body and extremity range of movement (ROM) movements.

In this instance the apparatus 200 includes components as previously described in communication with micro-processor 240.

Sensor 230 may take one of a multitude of forms.

The sensor 230 may comprise one or more of:

Accelerometer

Magnetometer

GPS Unit

EGC Sensor

Blood Pressure Sensor

Temperature Sensor

Range of Motion Sensor

By way of the communications module 260, signals from any one or more of the above described sensors or results processed from the signals by the micro-processor 240 may be communicated to other devices.

In one form, the other devices may include a portable digital communications device 261 or a data base 262.

In one form, the communications may comprise a signal in the form of a short range radio signal 263. The short range signal may be based on packet switched technology and may include Bluetooth and WiFi.

In other forms, the communications may comprise a signal in the form of a long range signal 264. The long range signal may be based on packet switched technology and may include cable based technologies such as TCP/IP which permit transmission over internet 265.

### Bidirectional Remote Communication Capability (H3)

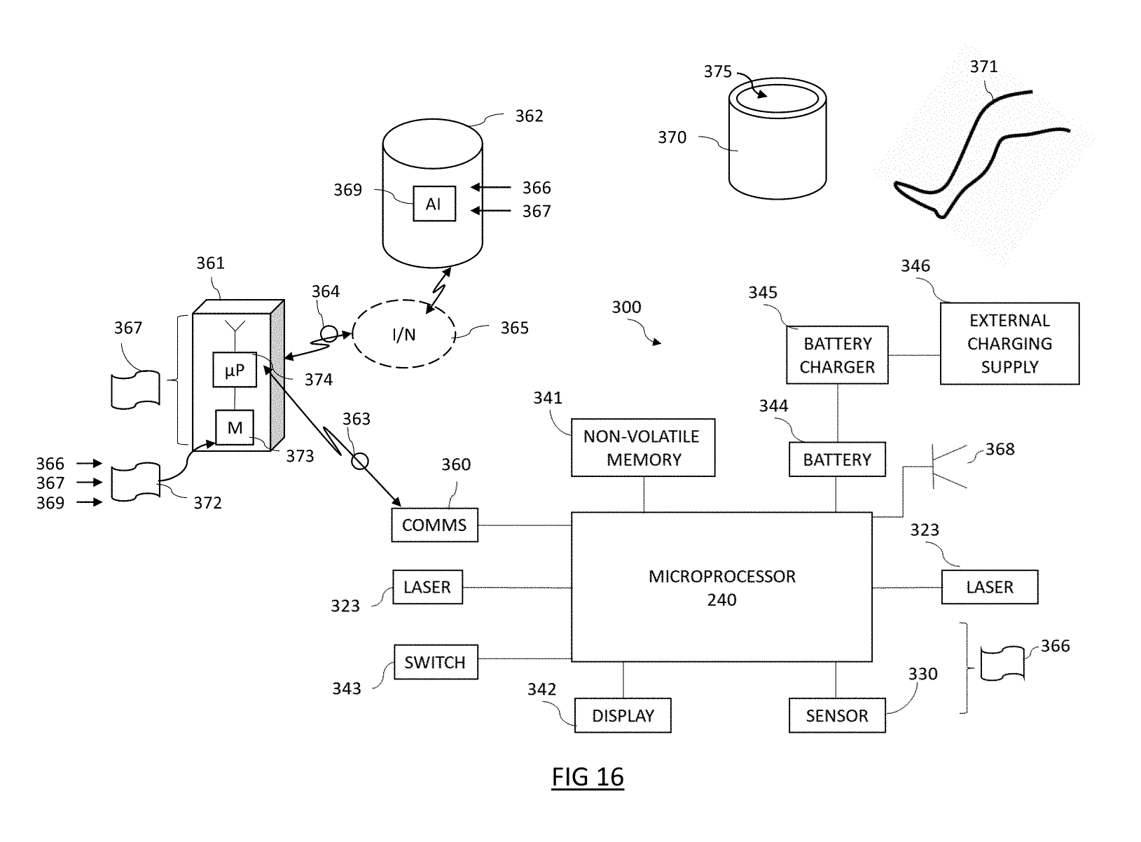

With reference to FIG. 16, there is illustrated apparatus 300 in accordance with a further embodiment of the present invention wherein like components are numbered as for earlier embodiments except in the 300 series.

In this instance communication from external sources such as database 362 or portable digital communications device 361 may proceed to communications device 360 by way of bidirectional long range signal 364 and/or bidirectional short range signal 363.

With the topology of apparatus 300, the system permits transmission of local sourced information 366 from sensor 330 out to portable digital communications device 361 or database 362.

The system also permits transmission of external sourced information 367 from database 362 and/or portable digital communications device 361 to processor 340. This external sourced information 367 may be used to enhance functionality of the apparatus 300. In a further form, the local sourced information 366 and the external sourced information 367 may also be used to enhance the functionality of apparatus 300.

In one form the functionality of apparatus 300 may be enhanced by incorporation of audio output 368 whereby audio instructions may be passed to a user of apparatus 300. In one form the constructions may derive from processing the local sourced information 366 through an Artificial Intelligence (AI) engine 369. In preferred forms the AI Engine 369 is associated with database 362 and makes use of local sourced information 366 and the external sourced information 367 thereby to provide intuitive and anticipatory commands and data to a user of apparatus 300.

### EXAMPLE 1

With further reference to FIG. 16, a particular form of sensor 330 in this instance in the form of a knee cover 370 is utilised by way of a first example of an application of an apparatus and method for measuring anatomical and physiological parameters of a body in accordance with an embodiment of the invention.

In this instance, the knee cover 370 has sensors in it. The knee cover 370 glides on over the knee 371, in a breathable, flexible, soft and washable material. The knee cover may consist of double layered material where by additional rehabilitation aids such as ice may be inserted in order to reduce the joint inflammation.

The double sleeve provides as inner sleeve which may also hold functions such as analgesic delivery to the joint or body part directly underneath the cover.

The inner sleeve may be made from a reusable material or disposable material, as part of a consumable part of the knee cover 370.

In preferred forms the knee cover 370 includes the apparatus 300 within it. The apparatus 300 communicates with portable digital communications device 361.

An application 372 stored in memory 373 processes the local sourced information 366 and the external sourced information 367 and also output from the AI engine 369 by way of processor 374 thereby to provide interactive guidance to a user of the knee cover 370.

The AI engine 369 will communicate with the knee cover 370 on a continuous basis and be interpreting and acting of the information from the sensors. The information may be stored and tracked within the portable digital communications device 361. The information may also be stored within a memory within the actual knee cover 370.

The application 372 permits the portable digital communications device 361 to operate as an intelligent, reactive assistant. It provides audio prompting by way of the audio output 368 and/or via audio output on the portable digital communications device 361.

Audio output 368 may also be transmitted through the knee cover 370 to be heard to come from the knee cover 370.

In a further form, sound may come from both the portable digital communications device 361 and knee cover 370 so both will have audible hardware available.

In a particular form the knee cover 370 may also include an inner sleeve 375 in the knee cover 370 that has the ability to release pain medication to the joint and deliver other therapies on instruction from and under the control of application 372.

The inner sleeve 375 of the knee cover 370 may have the ability to receive an ice pack therewithin for reacting to temperature variations as indicated by the sensors. In one form, the user may be signalled to obtain and insert an ice pack into the inner sleeve 375 of the knee cover 370.

In this instance the application 372 may be given a convenient name in order to assist the user to relate to the apparatus—for example ‘Halo Buddy’.

The knee cover 370, will interface with a portable digital communications device 361 associated with the user. In one form, the Halo buddy may be on continuously and ready to answer questions or ask questions based on the Biometric data it is receiving from the knee cover 370.

In one form the apparatus may be considered as an audible rehabilitation system. In one form, the system does not show exercises. Rather the system (termed HALO buddy) guides the user by means of audio prompts from the audio output 368 based on the movement it detects the user is doing, correcting and encouraging the user. The audible reactive technology acts like a personal physical therapist, nurse and surgeon.

In one form, the apparatus 300 or the portable digital communications device 361 may be implemented by way of a third party interactive assistant combined with an application. In this instance, this application may reside on a server or related device provided for use in association with the third party interactive assistant.

Audible two way feedback that is reactive to sensor information is a distinguishing characteristic of at least some forms of the present invention.

It may be thought of as Siri or Google Home for pre, peri and post-operative recovery.

In one form, the third party interactive assistant may be the ‘Google Home’ device marketed by Alphabet Corporation of the USA.

In an alternative form, the third party interactive assistant may be the ‘Siri Assistant’ device marketed by Apple Corporation in the USA.

In the case of the audio interactive assistant capability of example 1 an advantage is that it supports a user more in the capacity of a friend. Screen prompts or visual indications do not exhibit this capacity. The process of rehabilitation is thereby humanized and connects the professional to the user.

In a further form, the device is in the form of a cuff or cover that goes over or around a body part that is required to be supported and cared for post operatively. This can be for example a cover over the knee that fits the knee joint, filled with sensors that ‘subconsciously” monitor the patient. It is only when there is a requirement to interact, for example: An abnormal temperature or an exercise to be done that the Halo Buddy will ask the user a question to get more detail to interpret this. The Halo buddy can be asked any recovery based questions at any time by the user.

Stated another way in one form there is provided a device in the form of a simple knee brace that is filled with sensors.

Temperature

Motion

Pressure

Time

Pain

Device+Ai=Halo Buddy

The Ai is the buddy: an all knowing, all monitoring source for the user pre operative, peri operative and post operative.
1. Ai talks to the person in the tone and mannerism of a friend
2. English speaking first (multi-lingugal in the future)
3. Takes on Hospital and Surgeon specific information
4. Feeds back into a monitoring system. Ie. Temp control, pain monitoring, exercise so it can feedback to the patient as a Physio or Health professional would. Ie. You have done 1 km of walking today, so tomorrow your knee may be a little sore and even slightly swollen. Ai platform feeds back and reassures the patient, says to ice the knee, times the ice and sub-consciously runs health checks on the knee for the next 24, 48, 72 hrs. Any detected abnormal ranges, which are prolonged, would require a first check via Ai, then if persistent the Ai device would ring a health professional and give the monitored information. If requested they would schedule an appointment and then ask the patient to go in to see the health professional for a review.
5. We get the professional involved, only when there is a problem. Until then you are monitored by a feedback system that detects and checks, sometimes through asking you a question, sometimes through using the monitors in the knee brace.
6. Ai that learns when the patient likes to exercise, when they need a day off, when they need to be motivated to do their exercises.
7. A system that moves with the patient, ie. Exercise anywhere, no restrictions to sensors in the home, standing in front of the tv ect.
8. Ios and Android

In Use

A user may use the apparatus 300 as referenced under Example 1 above as follows:
a) In the case of wishing to provide use of the system in relation to the knee, a knee cover 370 will be provided and the user will slide the knee cover over the knee.
b) The knee cover 370 will include the apparatus 300 for monitoring anatomical and physiological parameters of the body of the user (in this case, the knee).
c) The user will download an application 372 to their portable digital communications device 361, in this case a smart phone.
d) The user will conduct an initial enrolment guided by the application 372.
e) Thereafter the user may proceed about their daily life.

In an alternative form the application download and intimal enrolment may occur in a clinical setting. For example, a health professional may conduct the application download and initial enrolment whilst the user is in the clinical setting (for example, a patient in a hospital or the like setting). It may then require the user to conduct a subsequent enrolment session or follow up enrolment session when they return to their home environment.

The user will receive audio prompts periodically as described above either to conduct specific exercises or audio prompts in the form of enquiries or encouragement.

The audio prompts may request the user to conduct certain activities—the example given above was to obtain an ice pack for insertion into the knee cover 370 to assist with rehabilitation/recovery resulting from local sourced information 366 sensed by the apparatus 300 and transmitted as a short range signal 363 in this instance to the portable digital communications device 361 and then via the long range signal 364 to the AI engine 369 whereby the AI engine 369 instigates the audio prompt to conduct the activity by way of a return long range signal and short range signal.

Other activities instigated by the AI engine 369 may include administration of pain medication as also described earlier.

How does it help the User

A user may benefit from the use of apparatus 300 as referenced under Example 1 above as follows:
Recovery
Reduce post operation complications
Support
Help when needed
Answer the questions as they come up, as and when they come up (don't have to save them up for the next surgeon check up)
Empowerment
Motivation
Knowledge to recover optimally
To feel cared for
Monitored
A friend
Reminders of post operation rules: 1 week until you can drive again.
Reminders (take out stitches, follow up needed, appointment reminder if already made in diary)
Recover is done with the patient, 24/7, monitored like never before
Base info: implant type, surgeon name that they can ask Halo buddy.

### SUMMARY

The apparatus 300 associated with the knee cover 370 as described with reference to example 1 may be used for other joints or regions of the body.

The AI engine 369 and system including apparatus 300 may be applied by way of further example to the shoulder and hip and knee.

The main thing is that we are bringing to life the concept of HUMANIZING the pre-operative, peri-operative and post-operative process through use of audible Ai, which will react when spoken to by the patient and when speaks to the patient when required to clarify information from which the Ai buddy receives from input from the sensors the patent has in the knee cover/brace that they are wearing.

Importantly the concept is also that the patient does not, in any way, think or know about the measures happening from the sensors inside their knee cover/brace, they are being monitored 24/7 and do not need to worry about the readings or check and record them at all. This is the beauty of Halo buddy, it does it all for you, and standardises recovery by primarily using objective data (heat,BP, range of motion ect) input, versus taking in subjective data from the patient. We could come up with a unique term to place in the continuation around “subconscious tracking or monitoring”, where by Ai is doing the monitoring versus the patient.

The reach that H3 will have great impact. We get to reach rural patients and 3rd world patients and offer them the knowledge of a Physical Therapist, Surgeon and Nurse on hand for them, to take home within the Halo buddy Ai system to help them recover optimally and feel supported throughout their recovery. We are also de-centralising health care by using this tech at home to assist with the patients versus utilising health care workers and taking up hospital beds/space as this will reduce hospital admissions and calls to hospitals given we can calm and support the patient exactly as something happens and we can monitor things like temperature and flag easily infections to fix prior to having to go back into surgery to fix these.

It will be appreciated by those skilled in the art that variations and modifications to the invention described herein will be apparent without departing from the spirit and scope thereof. The variations and modifications as would be apparent to persons skilled in the art are deemed to fall within the broad scope and ambit of the invention as herein set forth.

In particular, the knee cover 370 represents just one example of use of embodiments of the invention.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Throughout the specification and claims, unless the context requires otherwise, the term “substantially” or “about” will be understood to not be limited to the value for the range qualified by the terms.

It will be clearly understood that, if a prior art publication is referred to herein, that reference does not constitute an admission that the publication forms part of the common general knowledge in the art in Australia or in any other country.

1. Interactive support apparatus for measuring anatomical and physiological parameters of a body of a user and providing localised audio feedback to the user based on signals derived from measuring the anatomical and physiological parameters of the body;
said apparatus providing localised audio feedback to the user;
the feedback derived from local sourced information.

2. Interactive support apparatus of claim 1 wherein the feedback is additionally derived from external sourced information.

3. The apparatus of claim 2 wherein the external sourced information is determined from the local sourced information.

4. The apparatus of claim 2 wherein the external sourced information is determined from the local sourced information and from an AI Engine;
the AI engine acting on signals derived from the local sourced information and the external sourced information.

5. An interactive support method for measuring anatomical and physiological parameters of a body of a user and providing localised audio feedback to the user based on signals derived from measuring the anatomical and physiological parameters of the body;
said method providing localised audio feedback to the user;
the feedback derived from local sourced information.

6. The method of claim 5 wherein the feedback is additionally derived from external sourced information.

7. The method of claim 5 wherein the external sourced information is determined from the local sourced information.

8. The method of claim 6 wherein the external sourced information is determined from the local sourced information and from an AI Engine;
the AI engine acting on signals derived from the local sourced information and the external sourced information.

9. A method for measuring anatomical and physiological parameters of a body; said method comprising sensing at least one anatomical or physiological parameter of a body thereby to output a signal comprising a measurement of at least one anatomical or physiological parameter; said processor further including the step of transmitting the signal to an external location’ wherein the external location comprises a portable digital communications device; wherein the external location comprises a database; wherein the database includes a AI Engine; wherein the portable digital communications device is in bidirectional communication with the communications module;
wherein the database is in bidirectional communication with the communications module; said method further including an audio output device whereby the apparatus provides interactive audio guidance to a user.

10. The method of claim 9 wherein the signal is a short range signal.

11. The method of claim 9 wherein the sensor comprises one or more of accelerometer, Magnetometer, GPS Unit, EGC Sensor, Blood Pressure Sensor, Temperature Sensor, Range of Motion Sensor.

12. The method of claim 9 wherein the sensor is incorporated within a cuff.

13. The method of claim 9 wherein the sensor is incorporated in a cover.

14. The method of claim 13 wherein the sensor is incorporated in a joint cover.

15. The method of claim 13 wherein the sensor is incorporated in a knee cover.
